(19) United States
(12) Patent Application Publication
(10) Pub. No.: US 2022/0155835 A1
(43) Pub. Date: May 19, 2022

(54) BEDSIDE CARD DEVICE AND METHOD FOR CONTROLLING SAME

(71) Applicants: Beijing Zhongxiangying Technology Co., Ltd., Beijing (CN); BOE Technology Group Co., Ltd., Beijing (CN)

(72) Inventor: Li MA, Beijing (CN)

(21) Appl. No.: 17/515,216

(22) Filed: Oct. 29, 2021

(30) Foreign Application Priority Data
Nov. 17, 2020 (CN) 202022663933.2

(51) Int. Cl. G06F 1/26; G09F 9/30; F16M 11/22; F16M 13/02; G06F 3/14; H04W 4/80; G06F 1/18; G16H 40/67

(52) U.S. Cl. CPC G06F 1/26; G09F 9/30; F16M 11/22; G16H 40/67; G06F 3/14; H04W 4/80; G06F 1/188; F16M 13/022

(57) ABSTRACT

Provided is a bedside card device, including an electronic bedside card and a fixing assembly, wherein the electronic bedside card includes a housing, a display screen disposed on the housing, and a data connection circuit electrically connected to the display screen; the fixing assembly includes a bracket and a connecting member, the bracket is provided with a mounting slot having a shape conforming to the housing, and the connecting member is disposed on the bracket and is configured to connect to a mounting portion.

### CROSS-REFERENCE TO RELATED APPLICATION

This application is based on and claims priority to the Chinese Patent Application No. 202022663933.2, filed on Nov. 17, 2020 and entitled “BEDSIDE CARD DEVICE”, the disclosure of which is herein incorporated by reference in its entirety.

### TECHNICAL FIELD

The present disclosure relates to the field of medical devices, and in particular relates to a bedside card device and a method for controlling same.

### BACKGROUND

In hospital, a bedside card may be placed near each hospital bed. On the bedside card, information such as the identity data, diagnosis and nursing content of a patient may be recorded, so that the medical staff can distinguish between patients and learn the specific conditions of the respective patient, facilitating the treatment and nursing.

Currently, the bedside card is made of paper. After corresponding information has been filled, the card will be inserted into a slot near the bed. When it is necessary to change the nursing manner, the medical staff will replace the old card with a new card filled with necessary information.

### SUMMARY

The embodiments of the present disclosure provide a bedside card device and a method for controlling same. The technical solutions are as follows.

According to one aspect of the present disclosure, a bedside card device is provided, the bedside card device including an electronic bedside card and a fixing assembly; wherein

the electronic bedside card includes a housing, a display screen disposed on the housing, and a data connection circuit electrically connected to the display screen;

the fixing assembly includes a bracket and a connecting member, the bracket is provided with a mounting slot having a shape conforming to the housing, and the connecting member is disposed on the bracket and is configured to connect to a mounting portion.

In some embodiments, the fixing assembly includes a first conductive terminal disposed in the mounting slot and a data transmission line electrically connected to the first conductive terminal;

the electronic bedside card includes a second conductive terminal disposed outside the housing and electrically connected to the data connection circuit, and in a case that the housing is disposed in the mounting slot, the first conductive terminal is in contact with the second conductive terminal.

In some embodiments, the electronic bedside card includes a first wireless communication circuit disposed in the housing, the fixing assembly includes a second wireless communication circuit disposed on the bracket, and the first wireless communication circuit is configured to communicate with the second wireless communication circuit.

In some embodiments, the bracket includes a back plate connected to an end of the mounting slot, the housing includes a rear plate, the display screen is disposed on the rear plate, and in a case that the housing is disposed in the mounting slot, the rear plate and the back plate are opposed to each other;

the first wireless communication circuit includes a first near field communication circuit disposed on a side of the rear plate proximate to the display screen;

the second wireless communication circuit includes a second near field communication circuit disposed on the back plate.

In some embodiments, the fixing assembly includes a power supply circuitry disposed on the bracket;

the electronic bedside card includes a power source circuitry disposed on the housing and electrically connected to the display screen and the data connection circuit, and the power source circuitry matches with the power supply circuitry.

In some embodiments, the power supply circuitry includes a third conductive terminal disposed in the mounting slot and a power supply line electrically connected to the third conductive terminal;

the power source circuitry includes a fourth conductive terminal disposed outside the housing, and in a case that the housing is disposed in the mounting slot, the fourth conductive terminal is in contact with the third conductive terminal.

In some embodiments, the third conductive terminal is disposed at bottom of the mounting slot.

In some embodiments, the fixing assembly includes a data transmission line, and the third conductive terminal is electrically connected to the data transmission line.

In some embodiments, the power supply circuitry includes a battery connected to the power supply line.

In some embodiments, the power supply circuitry includes a wireless power supply circuit disposed on the bracket;

the power source circuitry includes a wireless charging circuit disposed on the housing.

In some embodiments, the bracket includes a back plate connected to the end of the mounting slot, and the wireless power supply circuit is disposed on the back plate.

In some embodiments, the housing includes a rear plate, the display screen is disposed on the rear plate, and in a case that the housing is disposed in the mounting slot, the rear plate and the back plate are opposed to each other;

the wireless charging circuit is disposed on a side of the rear plate proximate to the display screen.

In some embodiments, a material of the rear plate includes glass.

In some embodiments, the electronic bedside card further includes a camera disposed on the housing.

In some embodiments, the bedside card device further includes a connection string with one end connected to the housing, and the other end connected to the bracket.

In some embodiments, the connection string is provided with a conductive wire, the fixing assembly is provided with an alarm, and the alarm is electrically connected to the conductive wire.

In some embodiments, the connection string is in an extendable spiral shape.

In some embodiments, the connecting member is one selected from a screw connecting member or an adhesive plate.

In some embodiments, the connecting member includes a mechanical arm with one end connected to the bracket, and the other end configured to connect to the mounting portion.

According to another aspect of the present disclosure, a method for controlling a bedside card device is provided, the bedside card device including an electronic bedside card and a fixing assembly; wherein

the electronic bedside card includes a housing, a display screen disposed on the housing, and a data connection circuit electrically connected to the display screen;

the fixing assembly includes a bracket and a connecting member, the bracket is provided with a mounting slot having a shape conforming to the housing, and the connecting member is disposed on the bracket and is configured to connect to a mounting portion, and

wherein the method includes:

acquiring data to be transmitted by the data connection circuit; and

transmitting the data to be transmitted to the display screen by the data connection circuit.

### BRIEF DESCRIPTION OF THE DRAWINGS

To describe the technical solutions in the embodiments of the present disclosure more clearly, the following briefly introduces the accompanying drawings required for describing the embodiments. Apparently, the accompanying drawings in the following description show merely some embodiments of the present disclosure, and a person of ordinary skill in the art may derive other drawings from these accompanying drawings without creative efforts.

FIG. 1 is a schematic structural diagram of a bedside card device in the related art;

FIG. 2 is a schematic structural diagram of a bedside card device according to an embodiment of the present disclosure;

FIG. 3 is another schematic structural diagram of the bedside card device shown in FIG. 2;

FIG. 4 is a left side view of the bedside card device shown in FIG. 3;

FIG. 5 is a schematic structural diagram of another bedside card device according to an embodiment of the present disclosure;

FIG. 6 is a left side view of an electronic bedside card in the bedside card device shown in FIG. 5;

FIG. 7 is a schematic structural diagram of yet another bedside card device according to an embodiment of the present disclosure;

FIG. 8 is a schematic structural diagram of still another bedside card device according to an embodiment of the present disclosure;

FIG. 9 is a schematic stereoscopic structural diagram of the bedside card device shown in FIG. 5;

FIG. 10 is a schematic stereoscopic structural diagram of the bedside card device shown in FIG. 9 viewed from another direction;

FIG. 11 is a front view diagram of the bedside card device shown in FIG. 9.

With reference to these accompanying drawings, some specific embodiments of the present disclosure have been shown, and more detailed descriptions will be given hereinafter. These accompanying drawings and descriptions are not intended to limit the scope of the inventive concept of the present disclosure in any way, but only to illustrate the concepts of the present disclosure for those skilled in the art with reference to specific embodiments.

### DETAILED DESCRIPTION

For clearer descriptions of the objectives, technical solutions, and advantages of the present disclosure, the embodiments of present disclosure will be further described in details hereinafter with reference to the accompanying drawings.

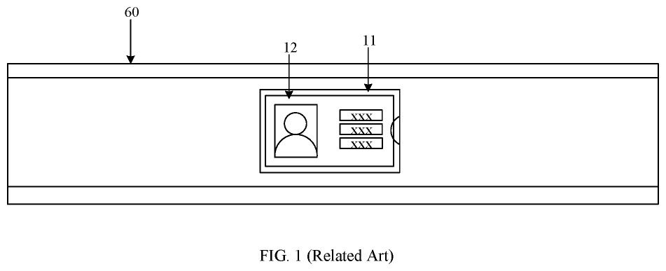

FIG. 1 is a schematic structural diagram of a bedside card device in the related art. The bedside card device used in the hospital includes a card slot 11 disposed on a device belt 60, and a bedside card made of plastic or paper. On the card, various related data of a patient are recorded.

However, when the data on the bedside card device need to be updated, the old bedside card will be discarded and a new one needs to be refilled, causing low update efficiency and waste of resources.

In addition, with the in-depth construction of medical informatization, currently, nosocomial information systems including Hospital Information System (HIS) and Nursing Information System (NIS) have been introduced in most domestic hospitals. However, the traditional bedside card cannot interact with such nosocomial information systems, causing disconnection of information in the medical system.

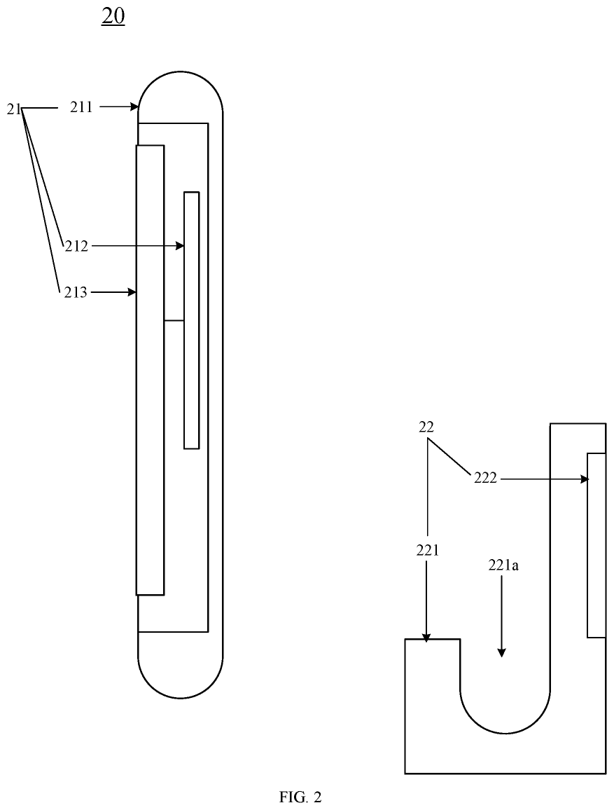

FIG. 2 is a schematic structural diagram of a bedside card device according to an embodiment of the present disclosure. The bedside card device 20 includes an electronic bedside card 21 and a fixing assembly 22.

The electronic bedside card 21 includes a housing 211, a data connection circuit 212 and a display screen 213. The display screen 213 is disposed on the housing 211, and the data connection circuit 212 is electrically connected to the display screen 213.

The fixing assembly 22 includes a bracket 221 and a connecting member 222, wherein the bracket 221 is provided with a mounting slot 221a having a shape conforming to the housing 211, and the connecting member 222 is disposed on the bracket 221 and is configured to connect to a mounting portion.

FIG. 2 shows the case in which the electronic bedside card 21 is not inserted into the fixing assembly 22. In this case, the electronic bedside card 21 may be operated by a medical staff, a patient, or a family member of a patient. For example, a medical staff may operate the electronic bedside card to update the related information of a patient, which is convenient and quick to operate.

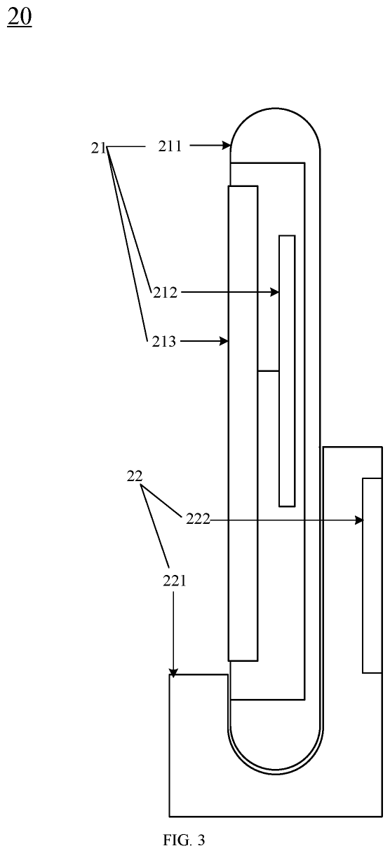

As shown in FIG. 3, which is a schematic structural diagram showing the case that the electronic bedside card 21 is inserted into the fixing assembly 22, the mounting slot 221a is engaged with the housing 211, so as to keep the housing 211 fixed.

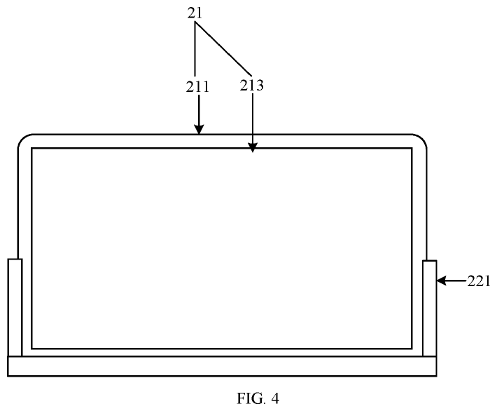

Exemplarily, as shown in FIG. 4, which is a left side view of the bedside card device shown in FIG. 3, the electronic bedside card 21 is disposed in the mounting slot of the bracket 221 of the fixing assembly, and the display screen 213 of the electronic bedside card 21 can display various information, so that the medical staff, the patient, and the family member of the patient can conveniently watch the display to learn the information.

Here, the data connection circuit may be disposed in the housing and establish communication with an external data provider, and update information (e.g., information related to the patient) displayed by the display screen based on the data communication with the data provider.

In summary, the embodiments of the present disclosure provide a bedside card device including an electronic bedside card and a fixing assembly. The fixing assembly may be connected to the mounting portion by a connecting member. The electronic bedside card can display information related to the patient by the display screen on the housing, and update the information displayed on the display screen conveniently by the data connection circuit. Furthermore, the electronic bedside card can be conveniently removed from the mounting slot of the fixing assembly, and is easy to operate, thereby providing a more convenient bedside card over the related art.

In some embodiments, the connecting member 222 is a screw connecting member (i.e., the fixing assembly 22 may be connected to the mounting portion by a screw), or the connecting member 222 is an adhesive plate. The mounting slot 221a may be an inserting slot.

The mounting portion may be a device belt, a wall, or other structures for mounting the fixing assembly in a hospital.

In some embodiments, the fixing assembly includes a power supply circuitry disposed on the bracket, the electronic bedside card includes a power source circuitry disposed on the housing, and the power source circuitry matches with the power supply circuitry. The power source circuitry may be electrically connected to both the display screen and the data connection circuit, so as to supply power to the display screen and the data connection circuit.

In the bedside card device provided by the embodiments of the present disclosure, configurations of the power source circuitry and the power supply circuitry may at least include the following two configurations.

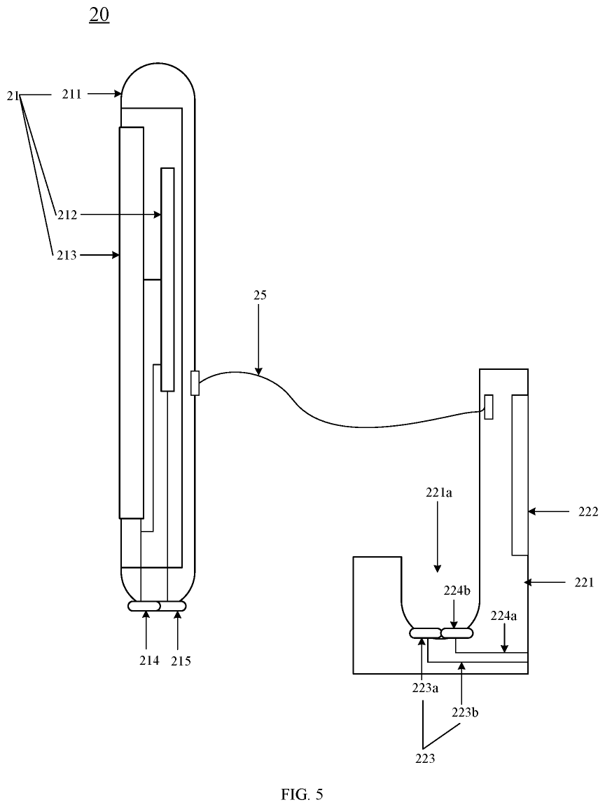

In a first configuration, as shown in FIG. 5, which is a schematic structural diagram of another bedside card device according to an embodiment of the present disclosure, a modified device based on the bedside card device shown in FIG. 2 is provided.

In some embodiments, the power supply circuitry 223 on the bracket 221 includes a third conductive terminal 223a and a power supply line 223b. The power supply line 223b is electrically connected to the third conductive terminal 223a, and the third conductive terminal 223a is disposed in the mounting slot 221a. The power supply line 223b may be connected to an external power line (e.g., a power line with a voltage ranging from 100 V to 220 V). Or, the power supply line 223b may be connected to a battery provided in the fixing assembly, which can supply power to the electronic bedside card as an emergency power source in a case that the external power line is powered off, thereby improving the usage time of the electronic bedside card when there is no external power supply.

The power source circuitry includes a fourth conductive terminal 214 disposed outside the housing 211, and in the case that the housing 211 is disposed in the mounting slot 221a, the fourth conductive terminal 214 is in contact with the third conductive terminal 223a on the bracket 221. The fourth conductive terminal 214 is electrically connected to the display screen 213 to supply power to the display screen 213. The fourth conductive terminal 214 may be further electrically connected to the data connection circuit 212 to supply power to the data connection circuit 212.

In addition, the power source circuitry of the electronic bedside card may further include a battery disposed inside the housing, and the fourth conductive terminal may be connected to the battery to charge the battery. The battery may be electrically connected to the display screen and the data connection circuit. It should be noted that the power source circuitry may control the internal connection manner of the electronic bedside card. For example, the electronic bedside card may have two operating modes. Under one operating mode, the electronic bedside card is disposed in the fixing assembly, the power source circuitry enables the fourth conductive terminal to be electrically connected to the display screen and the data connection circuit, so that the fixing assembly can supply power to the display screen and the data connection circuit in the electronic bedside card. In this case, the fixing assembly may further supply power to charge the battery in the electronic bedside card. Under the other operating mode, the electronic bedside card is disengaged from the fixing assembly, and in this case the power source circuitry may enable the battery to be electrically connected to the display screen and the data connection circuit, so that the battery in the electronic bedside card may supply power to the display screen and the data connection circuit.

In some embodiments, the third conductive terminal 223a is disposed at the bottom of the mounting slot 221a. In this case, when the housing of the electronic bedside card is inserted into the mounting slot, the fourth conductive terminal on the housing can easily contact the third conductive terminal.

In some embodiments, the fixing assembly 22 includes a data transmission line 224a and a first conductive terminal 224b. The data transmission line 224a is electrically connected to the first conductive terminal 224b, and the first conductive terminal 224b is disposed in the mounting slot 221a. The data transmission line 224a and the power supply line described above may be integrated into a type-c line. The data transmission lines in the fixing assembly may be connected to an external data transmission line, such as a data transmission line in the device belt of the hospital, and the data transmission line in the device belt may be connected to the Hospital Information System and the Nursing Information System of the hospital.

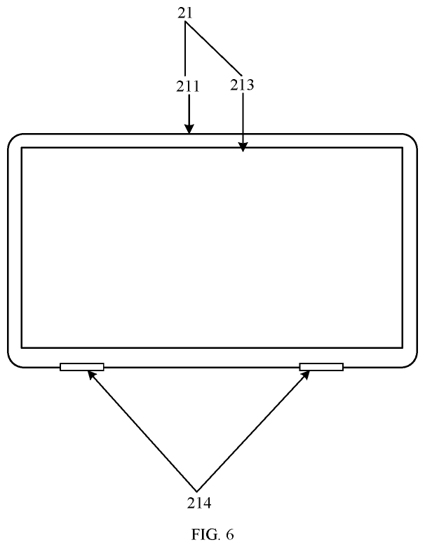

The electronic bedside card 21 includes a second conductive terminal 215 disposed outside the housing 211. The second conductive terminal 215 is electrically connected to the data connection circuit 212, and in the case that the housing 211 is disposed in the mounting slot 221a, the first conductive terminal 224b in the mounting slot 221a is in contact with the second conductive terminal 215. In this way, a wired data transmission function can be achieved via the second conductive terminal and the first conductive terminal in the mounting slot, thereby achieving the effect of updating the data displayed on the electronic bedside card via the data transmission line in the device belt based on the wired data transmission function. Here, the data connection circuit 212 may include a control circuit or a processor to process the received data. In addition, the data connection circuit 212 may include a memory to store the received data. As shown in FIG. 6, which is a left side view of the electronic bedside card 21 in the bedside card device shown in FIG. 5, there may be two fourth conductive terminals 214 (more fourth conductive terminals 214 may be disposed, e.g., four, five, six, etc., which is not limited in the embodiments of the present disclosure), and the two fourth conductive terminals 214 are disposed at the bottom edge of the electronic bedside card 21 and spaced apart from each other, so as to easily contact the third conductive terminals disposed at the bottom of the mounting slot.

Optionally, the second conductive terminal and the fourth conductive terminal of the electronic bedside card may be an integrated member. As such, the fourth conductive terminal may be electrically connected to the data transmission line. That is, the fourth conductive terminal may both act as a conductive terminal for supplying power and act as an interface for transmitting information, which can save space and simplify the structure of the electronic bedside card. In this case, the second conductive terminal and the fourth conductive terminal which are formed as an integrated member may be electrically connected to both the data connection circuit and the display screen, so as to supply power to the data connection circuit and the display screen, and transmit data to the data connection circuit.

Accordingly, the third conductive terminal and the first conductive terminal in the mounting slot of the fixing assembly may also be an integrated member. As such, the third conductive terminal may also be electrically connected to the data transmission line. That is, the third conductive terminal may both act as a conductive terminal for supplying power, and act as an interface for transmitting information.

In the bedside card devices according to the embodiments of the present disclosure, at least one of the first conductive terminal, the second conductive terminal, the third conductive terminal or the fourth conductive terminal may be a conductive contact pad, such as, a metal sheet (for example, a metal sheet including metal copper), and at least one of the above conductive terminals may be a metal contact bump or a metal tab, or the like.

The bedside card device shown in FIG. 5 has a contact power supply circuitry and a contact data transmission circuitry. Through these circuitries, when the electronic bedside card is placed in the mounting slot of the fixing assembly, the various functions such as the charging of the electronic bedside card or the data interaction can be achieved, which provides a more convenient and stable bedside card device that is easy to operate.

As shown in FIG. 5, the bedside card device 20 includes a connection string 25, one end of the connection string 25 is connected to the housing, and the other end of the connection string 25 is connected to the bracket. The connection string 25 may act as an anti-theft string to improve the security of the electronic bedside card. Exemplarily, the connection string 25 may be a wire rope.

Exemplarily, the connection string is provided with a conductive wire, the fixing assembly is further provided with an alarm, and the alarm is electrically connected to the conductive wire. In a case that the wire is cut off or forcibly broken, the alarm in the fixing assembly can sound an alarm, thereby further improving the security of the electronic bedside card and preventing it from being stolen. Certainly, the conductive wire in the connection string may also supply power to the electronic bedside card, which is not limited in the embodiments of the present disclosure.

In summary, the embodiments of the present disclosure provide a bedside card device including an electronic bedside card and a fixing assembly. The fixing assembly may be connected to the mounting portion by a connecting member. The electronic bedside card can display information related to the patient by the display screen on the housing, and update the information displayed on the display screen conveniently by the data connection circuit. Furthermore, the electronic bedside card can be conveniently removed from the mounting slot of the fixing assembly, and is easy to operate, thereby providing a more convenient bedside card over the related art.

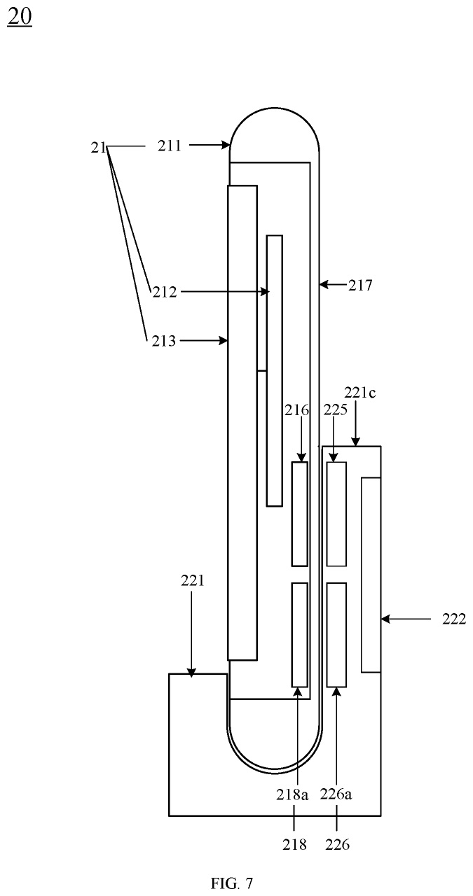

In a second configuration, as shown in FIG. 7, which is a schematic structural diagram of another bedside card device according to an embodiment of the present disclosure, a modified bedside card device based on the bedside card device shown in FIG. 2 is provided.

Here, the power supply circuitry includes a wireless power supply circuit 225. The wireless power supply circuit 225 may be connected to an external power line (e.g., a power line with a voltage ranging from 100 V to 220 V). Or, the wireless power supply circuit 225 may be connected to a battery provided in the fixing assembly.

The power source circuitry includes a wireless charging circuit 216 disposed on the housing 211. The wireless power supply circuit 225 may supply power to the wireless charging circuit 216 through a wireless charging technology. The wireless charging circuit 216 may be electrically connected to the display screen 213 and the data connection circuit 212.

In some embodiments, the bracket 221 includes a back plate 221c connected to an end of the mounting slot, and the wireless power supply circuit 225 is disposed on or in the back plate 221c.

In some embodiments, the housing 211 includes a rear plate 217. The display screen 213 is disposed on the rear plate 217, the wireless charging circuit 216 is disposed on a side of the rear plate 217 proximate to the display screen 213, and in a case that the housing 211 is disposed in the mounting slot 221a, the rear plate 217 and the back plate 221c are opposed to each other. With such structure, a distance between the wireless power supply circuit and the wireless charging circuit is relatively small, which can facilitate wireless charging of the electronic bedside card.

In some embodiments, the electronic bedside card 21 includes a first wireless communication circuit 218 disposed in the housing 211, the fixing assembly 22 includes a second wireless communication circuit 226 disposed on the bracket 221, and the first wireless communication circuit 218 is configured to communicate with the second wireless communication circuit 226.

The first wireless communication circuit 218 may include a first near field communication (NFC) circuit 218a, and the first near field communication circuit 218a may be disposed on a side of the rear plate 217 proximate to the display screen 213.

The second wireless communication circuit 226 may include a second near field communication circuit 226a, and the second near field communication circuit 226a may be disposed on the back plate 221c. A wireless data transmission function between the electronic bedside card and the fixing assembly can be achieved through the first near field communication circuit and the second near field communication circuit. The second near field communication circuit in the fixing assembly may be connected to an external data transmission line, such as a data transmission line in the device belt of the hospital, and the data transmission line in the device belt may be connected to the Hospital Information System and the Nursing Information System of the hospital, thereby achieving the technical effect of updating the data displayed on the electronic bedside card by the data transmission line in the device belt based on the wireless data transmission function. Certainly, the first wireless communication circuit and the second wireless communication circuit may also communicate through other wireless communication means, such as WiFi, Bluetooth, or the like, which is not limited in the embodiments of the present disclosure.

Optionally, a material of the rear plate 217 may include glass. The rear plate made of glass can reduce the interference on wireless charging, and improve the efficiency of wireless charging and energy utilization.

With the wireless charging configuration described above, the bedside card device provided by the embodiments of the present disclosure can eliminate the exposure of conductive terminal, so that the entire structure of the electronic bedside card is more robust and durable, and the housing has a more excellent sealing performance, thereby facilitating the procedures such as cleaning, disinfection and the like.

In the bedside card devices provided in the embodiments of the present disclosure, the performances of the electronic bedside card in terms of the interaction and convenience can be improved. With the features of the utilization of the screen, the contact charging and the movability, the electronic bedside card becomes a platform for serving the patients, which improves the patient service level of the hospital, and reduces the walking amount and workload of nursing staff, thereby facilitating the functions of check-in education, preoperative communication, notice of precautions and the like by autonomous services, and providing a better service to the patients.

In the bedside card devices provided in the embodiments of the present disclosure, the display screen in the electronic bedside card may be a liquid crystal display screen, an organic light emitting diode display screen, or other types of display screens.

In summary, the embodiments of the present disclosure provide a bedside card device including an electronic bedside card and a fixing assembly. The fixing assembly may be connected to the mounting portion by a connecting member. The electronic bedside card can display information related to the patient by the display screen on the housing, and update the information displayed on the display screen conveniently by the data connection circuit. Furthermore, the electronic bedside card can be conveniently removed from the mounting slot of the fixing assembly, and is easy to operate, thereby providing a more convenient bedside card over the related art.

The two kinds of bedside card devices described above provide a wired charging configuration and a wireless charging configuration respectively, and further provide a wired data transmission configuration and a wireless data transmission configuration respectively. As such, the bedside card devices according to the embodiments of the present disclosure may at least have one of the above power source configurations and one of the above data transmission configurations.

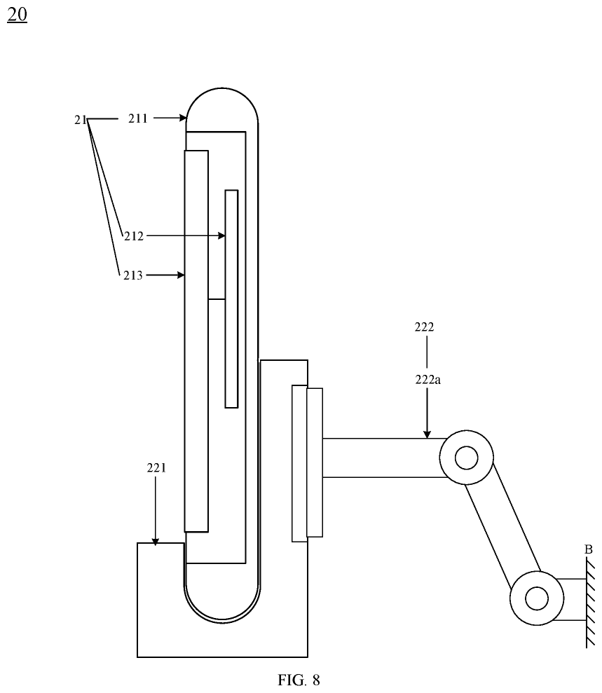

As shown in FIG. 8, which is a schematic structural diagram of another bedside card device according to an embodiment of the present disclosure, a modified bedside card device based on the bedside card device shown in FIG. 2 is provided.

Here, the connecting member 222 includes a mechanical arm 222a, one end of the mechanical arm 222a is connected to the bracket 221, and the other end of the mechanical arm 222a is connected to a fixing structure B. Exemplarily, the fixing structure B is a wall. The mechanical arm 222a may be moved along multiple directions to greatly increase the movement range of the electronic bedside card, which is more convenient for the healthcare staff or patients to watch and use the electronic bedside card. Exemplarily, the position of the electronic bedside card is adjusted by the mechanical arm, so that the patient can use the electronic bedside card in bed.

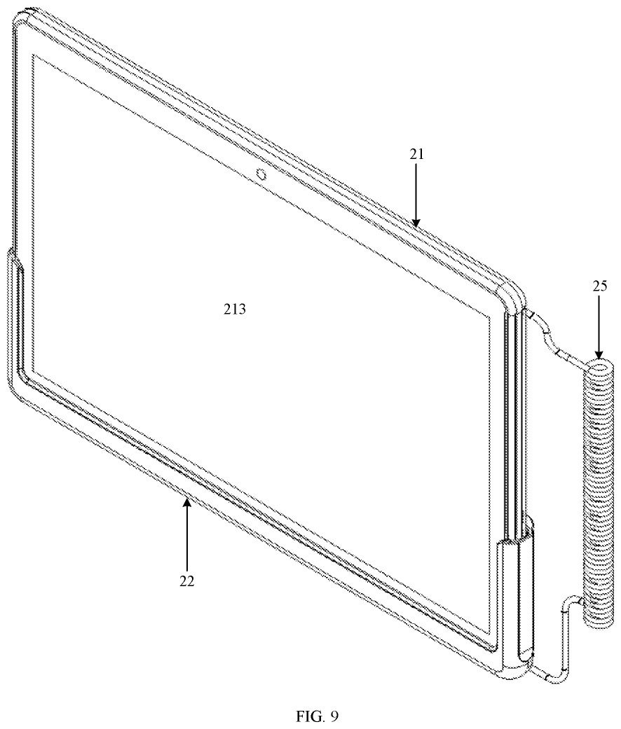

FIG. 9 is a schematic stereoscopic structural diagram of the bedside card device shown in FIG. 5. FIG. 9 shows the case in which the electronic bedside card 21 is in the mounting slot of the fixing assembly 22. The display screen 213 on the electronic bedside card 21 may display various information, and an edge the mounting slot adjacent the display screen can be relatively low, so as to avoid the blocking of the display screen 213.

The electronic bedside card 21 is connected to the bracket of the fixing assembly 22 by a connection string 25. The connection string 25 may be a spiral wire, and may be in an extendable spiral shape, so as to facilitate the contraction and extension of the connection string 25 and enhance the activity range of the electronic bedside card 21.

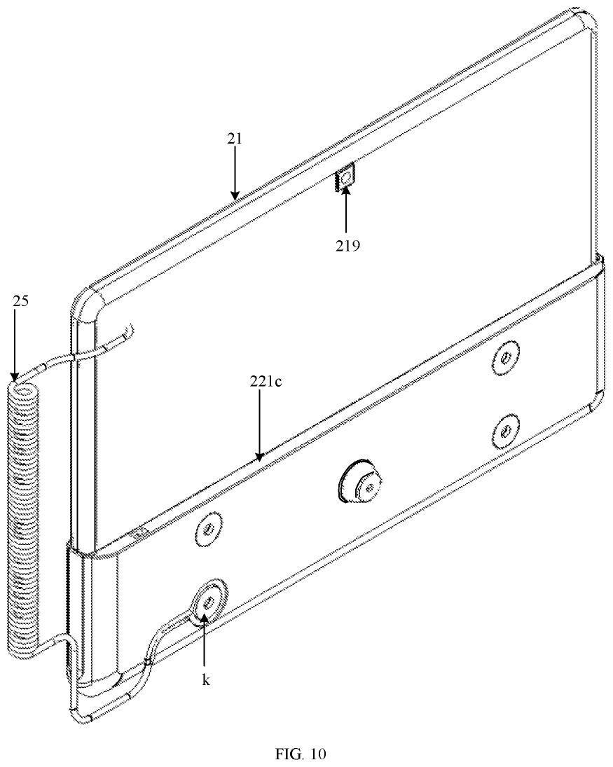

As shown in FIG. 10, which is a schematic stereoscopic structural diagram of the bedside card device shown in FIG. 9 viewed from another direction, the back plate 221c of the fixing assembly 22 is provided with a connector k. One end of the connection string 25 may be connected to the connector k, and the other end of the connection string 25 is connected to the back side of the housing of the electronic bedside card 21 (the back side of the housing being a side opposite to its display screen). The connection string 25 is connected to both the electronic bedside card 21 and the fixing assembly 22 to achieve an anti-theft or other functions.

It should be noted that the connection string 25 is an optional structure. That is, the electronic bedside card 21 and the fixing assembly 22 may be not connected by the connection string 25.

In some embodiments, the electronic bedside card 21 further includes a camera 219, and the camera 219 may be disposed on the housing. The camera 219 may be disposed on the same side as the display screen of the housing, or disposed on a side opposite to the display screen of the housing (i.e., the camera being disposed on the back of the housing), or each of the two sides of the housing may be provided with one camera 219. FIG. 10 shows a configuration of the camera 219 disposed on the side distal from the display screen of the housing. The camera may be a rear camera.

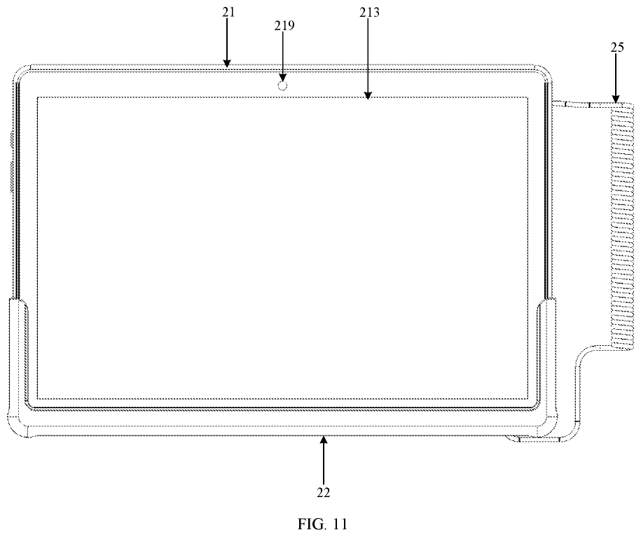

As shown in FIG. 11, which is a front view diagram of the bedside card device shown in FIG. 9, a camera 219 is disposed on the same side as the display screen 213 of the housing of the electronic bedside card 21, and the camera on the same side as the display screen 213 is a front camera.

According to another aspect of the present disclosure, a method for controlling a bedside card device is further provided, and the method may be applied to the bedside card device provided by the embodiments described above. The method includes:

1) acquiring data to be transmitted by a data connection circuit; and

2) transmitting the data to be transmitted to the display screen by the data connection circuit. The display screen may display the data to be transmitted.

In this way, the data displayed on the display screen can be conveniently updated by the data connection circuit in the bedside card device. With reference to the contents of the embodiments described above, the data connection circuit may acquire the data to be transmitted in a wired or wireless manner, which is not limited in the embodiments of the present disclosure.

The terms “at least one of A and B” in the present disclosure are merely a relationship describing the associated object, indicating that there may be three relationships: A existing alone, A and B existing at the same time, and B existing alone. In the same way, “at least one of A, B, and C” indicates that there may be seven relationships: A existing alone, B existing alone, C existing alone, A and B existing at the same time, A and C existing at the same time, C and B existing at the same time, and A, B, and C existing at the same time. In the same way, “at least one of A, B, C, and D” indicates that there may be fifteen relationships: A existing alone, B existing alone, C existing alone, D existing alone, A and B existing at the same time, A and C existing at the same time, A and D existing at the same time, C and B existing at the same time, D and B existing at the same time, C and D existing at the same time, A, B and C existing at the same time, A, B and D existing at the same time, A, C and D existing at the same time, B, C and D existing at the same time, and A, B, C and D existing at the same time.

In the present disclosure, the terms “first”, “second”, “third” and “fourth” are merely used for the purpose of description and should not be understood as indicating or implying relative importance. The term “multiple” refers to two or more, unless specially defined otherwise.

Described above are merely optional embodiments of the present disclosure, and are not intended to limit the present disclosure. Any modifications, equivalent replacements, improvements and the like made within the spirit and principles of the present disclosure should be included within the scope protection of the present disclosure.

In some embodiments, a bedside card device is provided, the bedside card device includes an electronic bedside card and a fixing assembly;

the electronic bedside card includes a housing, a data connection circuit, and a display screen disposed on the housing, and the data connection circuit is electrically connected to the display screen;

the fixing assembly includes a bracket, the bracket is provided with a mounting slot and a connecting member configured to connect to a mounting portion, and the housing has a shape matching with the mounting slot.

In some embodiments, the fixing assembly includes a power supply circuitry disposed on the bracket, the electronic bedside card includes a power source circuitry disposed on the housing, and the power source circuitry matches with the power supply circuitry.

In some embodiments, the power supply circuitry on the bracket includes a first conductive contact disposed in the mounting slot and a power supply line electrically connected to the first conductive contact;

the power source circuitry includes a second conductive contact disposed outside the housing, and in a case that the housing is disposed in the slot, the second conductive contact is in contact with the first conductive contact.

In some embodiments, the first conductive contact is disposed at the bottom of the mounting slot.

In some embodiments, the fixing assembly includes a data transmission line and a third conductive contact disposed in the mounting slot, and the data transmission line is electrically connected to the third conductive contact;

the electronic bedside card includes a fourth conductive contact disposed outside the housing, the fourth conductive contact is electrically connected to the data connection circuit, and in a case that the housing is disposed in the mounting slot, the third conductive contact is contacted with the fourth conductive contact.

In some embodiments, the power supply circuitry includes a wireless power supply circuit; and

the power source circuitry includes a wireless charging circuit disposed on the housing.

In some embodiments, the bracket includes a back plate connected to an end of the mounting slot, and the wireless power supply circuit is disposed on the back plate.

In some embodiments, the housing includes a rear plate, the display screen is disposed on the rear plate; the wireless charging circuit is disposed on a side of the rear plate proximate to the display screen, and in a case that the housing is disposed in the mounting slot, the rear plate and the back plate are opposed to each other.

In some embodiments, the electronic bedside card includes a first near field communication circuit disposed on a side of the rear plate proximate to the display screen; and

the fixing assembly includes a second near field communication circuit disposed on the back plate.

In some embodiments, a material of the rear plate includes glass.

In some embodiments, the bedside card device includes a connection string, with one end of the connection string connected to the housing, and the other end connected to the bracket.

In some embodiments, the connection string is provided with a conductive wire, the fixing assembly is further provided with an alarm, and the alarm is electrically connected to the conductive wire.

In some embodiments, the bedside card device includes a mechanical arm, one end of the mechanical arm, acting as a mounting portion, is connected to the connecting member of the bracket, and the other end of the mechanical arm is connected to a fixing structure.

The technical solutions provided by the embodiments of the present disclosure herein at least achieve the following benefits.

The bedside card devices provided by the embodiments of the present disclosure include an electronic bedside card and a fixing assembly. The fixing assembly may be connected to the mounting portion by a connecting member. The electronic bedside card can display information related to the patient by the display screen on the housing, and update the information displayed on the display screen conveniently by the data connection circuit. Furthermore, the electronic bedside card can be conveniently removed from the mounting slot of the fixing assembly, and is easy to operate, thereby providing a more convenient bedside card over the related art.

What is claimed is:

1. A bedside card device, comprising an electronic bedside card and a fixing assembly, wherein the electronic bedside card comprises a housing, a display screen disposed on the housing, and a data connection circuit electrically connected to the display screen;
the fixing assembly comprises a bracket and a connecting member, the bracket is provided with a mounting slot having a shape conforming to the housing, and the connecting member is disposed on the bracket and is configured to connect to a mounting portion.

2. The bedside card device according to claim 1, wherein the fixing assembly comprises a first conductive terminal disposed in the mounting slot and a data transmission line electrically connected to the first conductive terminal;
the electronic bedside card comprises a second conductive terminal disposed outside the housing and electrically connected to the data connection circuit, and in a case that the housing is disposed in the mounting slot, the first conductive terminal is in contact with the second conductive terminal.

3. The bedside card device according to claim 1, wherein the electronic bedside card comprises a first wireless communication circuit disposed in the housing, the fixing assembly comprises a second wireless communication circuit disposed on the bracket, and the first wireless communication circuit is configured to communicate with the second wireless communication circuit.

4. The bedside card device according to claim 3, wherein the bracket comprises a back plate connected to an end of the mounting slot, the housing comprises a rear plate, the display screen is disposed on the rear plate, and in a case that the housing is disposed in the mounting slot, the rear plate and the back plate are opposed to each other;
the first wireless communication circuit comprises a first near field communication circuit disposed on a side of the rear plate proximate to the display screen;
the second wireless communication circuit comprises a second near field communication circuit disposed on the back plate.

5. The bedside card device according to claim 1, wherein the fixing assembly comprises a power supply circuitry disposed on the bracket;
the electronic bedside card comprises a power source circuitry disposed on the housing and electrically connected to the display screen and the data connection circuit, and the power source circuitry matches with the power supply circuitry.

6. The bedside card device according to claim 5, wherein the power supply circuitry comprises a third conductive terminal disposed in the mounting slot and a power supply line electrically connected to the third conductive terminal;
the power source circuitry comprises a fourth conductive terminal disposed outside the housing, and in a case that the housing is disposed in the mounting slot, the fourth conductive terminal is in contact with the third conductive terminal.

7. The bedside card device according to claim 6, wherein the third conductive terminal is disposed at bottom of the mounting slot.

8. The bedside card device according to claim 6, wherein the fixing assembly comprises a data transmission line, and the third conductive terminal is electrically connected to the data transmission line.

9. The bedside card device according to claim 6, wherein the power supply circuitry comprises a battery connected to the power supply line.

10. The bedside card device according to claim 5, wherein the power supply circuitry comprises a wireless power supply circuit disposed on the bracket;
the power source circuitry comprises a wireless charging circuit disposed on the housing.

11. The bedside card device according to claim 10, wherein the bracket comprises a back plate connected to the end of the mounting slot, and the wireless power supply circuit is disposed on the back plate.

12. The bedside card device according to claim 11, wherein the housing comprises a rear plate, the display screen is disposed on the rear plate, and in a case that the housing is disposed in the mounting slot, the rear plate and the back plate are opposed to each other;
the wireless charging circuit is disposed on a side of the rear plate proximate to the display screen.

13. The bedside card device according to claim 12, wherein a material of the rear plate comprises glass.

14. The bedside card device according to claim 1, wherein the electronic bedside card further comprises a camera disposed on the housing.

15. The bedside card device according to claim 1, further comprising a connection string with one end connected to the housing, and the other end connected to the bracket.

16. The bedside card device according to claim 15, wherein the connection string is provided with a conductive wire, the fixing assembly is provided with an alarm, and the alarm is electrically connected to the conductive wire.

17. The bedside card device according to claim 15, wherein the connection string is in an extendable spiral shape.

18. The bedside card device according to claim 1, wherein the connecting member is one selected from a screw connecting member or an adhesive plate.

19. The bedside card device according to claim 1, wherein the connecting member comprises a mechanical arm with one end connected to the bracket, and the other end configured to connect to the mounting portion.

20. A method for controlling a bedside card device, the bedside card device comprising an electronic bedside card and a fixing assembly,
wherein the electronic bedside card comprises a housing, a display screen disposed on the housing, and a data connection circuit electrically connected to the display screen;
the fixing assembly comprises a bracket and a connecting member, the bracket is provided with a mounting slot having a shape conforming to the housing, and the connecting member is disposed on the bracket and is configured to connect to a mounting portion, and
wherein the method comprises:
acquiring data to be transmitted by the data connection circuit; and
transmitting the data to be transmitted to the display screen by the data connection circuit.
